(19) United States
(12) Patent Application Publication
(10) Pub. No.: US 2017/0087265 A1
(43) Pub. Date: Mar. 30, 2017

(54) ANTIMICROBIAL AIR SANITIZER COMPOSITIONS AND THEIR USE

(71) Applicant: AIR SANZ HOLDINGS PTY LTD, Donvale (AU)

(72) Inventors: Paul Geoffrey SMYTH, Murarrie (AU); Roger Gordon WHITE, Mansfield (AU)

(21) Appl. No.: 15/126,469

(22) PCT Filed: Mar. 17, 2015
(86) PCT No.: PCT/AU2015/000152

§ 371 Date: Sep. 15, 2016

(30) Foreign Application Priority Data
Mar. 17, 2014 (AU) 2014900913

(51) Int. Cl. A61L 9/01; A61L 9/14; A61L 9/015; A01N 33/12

(52) U.S. Cl. CPC A61L 9/01; A01N 33/12; A61L 9/14; A61L 9/015

(57) ABSTRACT

The present invention relates to antimicrobial sanitizing compositions for use as air sanitizers and deodorisers. The sanitizing compositions comprise a combination of an alkoxy silyl ammonium compound, a benzalknonium chloride or benzethonium chloride and a polymeric biguanide in a glycol carrier. Methods of use of the compositions are also described.

### FIELD OF THE INVENTION

The present invention relates to antimicrobial air sanitizing compositions. The sanitizing compositions comprise a combination of three ammonium compounds in a glycol solvent. Methods of use of the compositions are also described.

### BACKGROUND OF THE INVENTION

The environment in which humans carry out activities and congregate for work, pleasure, leisure or for health reasons is an environment where microorganisms, such as bacteria, viruses and fungi, are present. These environments can put humans at risk of infection with such microorganisms. Serious epidemics of infection with viruses such as flu viruses, can be readily spread in environments such as hospitals, medical centres, airports, schools, child care centres, nursing homes, gyms, sports clubs, shopping centres and the like.

Air sanitizers and deodorizers in common use are often predominantly composed of fragrances that mask odours but do not effectively remove microorganisms from the environment. Some air sanitizers use toxic chemicals to kill microorganisms in the air and therefore are only useful in limited environments. In yet other air sanitizing products, the rate of distribution, for example, by evaporation or diffusion can be difficult to control.

There is a need for air sanitizing compositions that are non-toxic or have low toxicity, remove microbes from the air and their diffusion is easy to control.

The present invention is predicated at least in part on the discovery that combinations of specific quaternary ammonium compounds in glycol carriers can provide non-toxic air sanitizing activity with controlled release when used in air sanitizing mechanical distribution apparatus.

### SUMMARY OF THE INVENTION

In a first aspect of the invention there is provided an air sanitizing composition comprising:
(i) an alkoxy silyl ammonium film-forming compound;
(ii) a benzalkonium or benzethonium chloride;
(iii) a polymeric biguanide; and
(iv) a glycol carrier.

In some embodiments, the composition further comprises a cross-linking agent. In some embodiments, the composition further comprises a fragrance.

In another aspect of the present invention there is provided a method of sanitizing and/or deodorizing air in an enclosed environment comprising dispersing the composition of the invention into the environment.

In yet another aspect of the invention there is provided the use of the composition of the invention as an air sanitizer and/or deodorizer.

In some embodiments, the composition of the invention is dispersed from an air purifying apparatus.

### DESCRIPTION OF THE INVENTION

In a first aspect of the invention there is provided an air sanitizing composition comprising:
(v) an alkoxy silyl ammonium film-forming compound;
(vi) a benzalkonium or benzethonium chloride;
(vii) a polymeric biguanide; and
(viii) a glycol carrier.

In some embodiments, the alkoxy silyl ammonium film-forming agent is a compound of formula (I):

[(R₁O)₃Si-A-N⁺(W)(X)(Y)] M⁻ (I)

wherein each R₁ is independently selected from hydrogen and —C₁₋₆alkyl;

A is a C₁₋₆alkylene group;

W and X are independently selected from —C₁₋₆alkyl;

Y is a C₁₀₋₂₀alkyl group; and

M is an anionic counterion.

In particular embodiments, one or more of the following applies:

R₁ is hydrogen, methyl or ethyl, especially methyl or ethyl, more especially methyl;

A is a C₂₋₄alkylene group, especially —CH₂CH₂CH₂—;

W and X are independently selected from methyl and ethyl, especially methyl;

Y is C₁₀₋₁₈alkyl; especially C₁₀alkyl or C₁₈alkyl, more especially C₁₈alkyl; and

M is selected from F⁻, Cr, Br⁻ and I—, especially Cl⁻.

In particular embodiments, the alkoxy silyl ammonium film-forming compound of formula (I) is selected from 1-octadecanaminium-N,N-dimethyl-N-[3-trimethoxysilyl(propyl)]chloride (also known as 3-trimethoxysilylpropyl-N,N-dimethyl-N-octadecyl ammonium chloride), 3-triethoxysilylpropyl-N,N-dimethyl-N-octadecyl ammonium chloride, 3-triethoxysilylpropyl-N,N-dimethyl-N-isodecyl ammonium chloride or 3-trimethoxysilylpropyl-N,N-dimethyl-N-isodecyl ammonium chloride.

In some embodiments, the alkoxy silyl ammonium film-forming compound is present in the composition in an amount of 0.1% to 1.5% w/w, especially about 0.5% to 1.0% w/w, more especially about 0.5 to 0.9% w/w of the composition.

The benzalkonium or benzethionium chloride compound is a compound of formula (II):

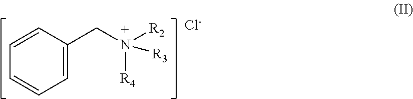

wherein R₂ and R₃ are independently selected from C₁₋₆alkyl and

R₄ is C₈₋₁₈alkyl or —(CH₂CH₂O)₂[4-(1-dimethyl-3-dimethylbutyl)phenyl].

In particular embodiments, one or more of the following applies:

R₂ and R₃ are independently selected from methyl or ethyl, especially methyl; and

R₄ is selected from C₈, C₁₀, C₁₂, C₁₄, C₁₆ or C₁₈ alkyl or mixtures thereof or —(CH₂CH₂O)₂[4-(1-dimethyl-3-dimethylbutyl)phenyl].

The benzalkonium or benzthionium chloride compound is present in an amount in the range of about 0.1% to 1.0% w/w of the composition, especially about 0.1% to about 0.5% w/w, more especially about 0.1 to 0.2% w/w of the composition.

In some embodiments, the polymeric biguanide is a compound of formula (III):

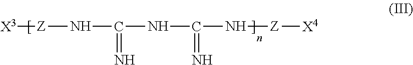

wherein Z is absent or an organic divalent bridging group and each Z may be the same or different throughout the polymer; n is at least 3, preferably 5 to 20 and X³ and X⁴ are independently selected from —NH₂, —NH—C(═NH)—NH—CN, optionally substituted alkyl, optionally substituted cycloalkyl, optionally substituted aryl, optionally substituted heterocyclyl and optionally substituted heteroaryl; or a pharmaceutically acceptable salt thereof. Preferably, the molecular weight of the polymeric compound is at least 1,000 amu, more preferably between 1,000 amu and 50,000 amu. In a single composition, n may vary providing a mixture of polymeric biguanides.

The above polymeric biguanide compounds and methods for their preparation are described in, for example, U.S. Pat. No. 3,428,576 and East et. al., 1997.

In some embodiments, the polymeric biguanide for use in the invention are polymeric alkylene biguanides of the following formula (IV):

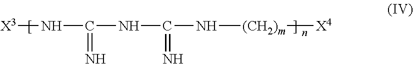

wherein n is an integer from 3 to 500, m is 1 to 10, especially 3 or 6, and X³ and X⁴ are independently selected from —NH₂, —NH—C(═NH)—NH—CN, optionally substituted alkyl, optionally substituted cycloalkyl, optionally substituted aryl, optionally substituted heterocyclyl and optionally substituted aryl or a pharmaceutically acceptable salt thereof. In particular embodiments, n has an average value of 3 to 15, more especially 3 to 12. A suitable polymeric biguanide is sold under the trade name Cosmocil CQ™ (Lonza).

The polymeric biguanide is present in the composition in an amount in the range of about 0.05% to 1.0% w/w of the composition, more especially about 0.1% to 0.5% w/w of the composition, more especially about 0.1% to about 0.3% w/w of the composition.

The carrier is a glycol carrier. In some embodiments, the glycol carrier is selected from dipropylene glycol, tripropylene glycol, propylene glycol and polyethylene glycol. In particular embodiments, the glycol carrier is dipropylene glycol. Dipropylene glycol is an isomeric mixture of glycol compounds including 4-oxa-2,6-heptandiol, 2-(2-hydropypropoxy)propan-1-ol and 2-(2-hydroxy-1-methylethyoxy)propan-1-ol.

The carrier is present in an amount in the range of 40% to 99% w/w of the composition, especially 40% to 70% w/w of the composition, more especially about 50% to 65% w/w of the composition.

The compositions of the invention may further comprise fragrances. The fragrances may be any fragrance desired to provide the desired background smell. Suitable fragrances include synthetic fragrances, natural fragrances, essential oils and mixtures thereof. Some suitable fragrances include citrus oils such as orange, lime, lemon, tangerine, grapefruit, floral scents such as jasmine, rose, gardenia; fruit scents such as mango, banana, raspberry, apricot, apple; spicy fragrances such as vanilla, cinnamon, cloves, nutmeg; fragrance oils such as ylang ylang, sandalwood, cedarwood, lemon grass, rosehip, peppermint and eucalyptus or mixtures of any of these fragrances.

The fragrance may be present in the composition in any amount suitable to provide the level of air fragrance desired. Typically, the fragrance is present in an amount of 20% to 60% w/w of the composition, especially about 30% to 50% w/w of the composition, more especially 35% to 45% w/w of the composition.

In particular embodiments, the composition is in the form of a viscous liquid.

In some embodiments, the composition is dispersed into the air by diffusion or evaporation. In some embodiments, the composition is dispersed in a device that provides a mist or fog or spray or purifies air and disperses the composition in its air flow.

Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, the term “alkyl” refers to a straight chain or branched saturated hydrocarbon group having 1 to 20 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl, decyl, isodecyl, undecyl, dodecyl and the like.

As used herein, the term “cycloalkyl” refers to a saturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentyl, cyclohexyl, cycloheptyl and cyclooctyl.

As used herein, the term “aryl” is intended to mean any stable, monocyclic, bicyclic or tricyclic carbon ring system of up to 7 atoms in each ring, wherein at least one ring is aromatic. Examples of such aryl groups include, but are not limited to, phenyl, naphthyl, tetrahydronaphthyl, indanyl, fluorenyl, phenanthrenyl, biphenyl and binaphthyl.

As used herein, the term “alkylene” refers to a divalent saturated hydrocarbon chain having 1 to 6 carbon atoms. Where appropriate, the alkylene group may have a specified number of carbon atoms, for example, C₁₋₆alkylene includes alkylene groups having 1, 2, 3, 4, 5 or 6 carbon atoms in a linear arrangement. Examples of suitable alkylene groups include, but are not limited to, —CH₂—, —CH₂CH₂—, —CH₂CH₂CH₂—, —CH₂CH₂CH₂CH₂—, —CH₂CH₂CH₂CH₂CH₂— and —CH₂CH₂CH₂CH₂CH₂CH₇—.

The term “heterocyclic” or “heterocyclyl” as used herein, refers to a cyclic hydrocarbon in which one to four carbon atoms have been replaced by heteroatoms independently selected from the group consisting of N, N(R), S, S(O), S(O)₂ and O. A heterocyclic ring may be saturated or unsaturated but not aromatic. A heterocyclic group may also be part of a spirocyclic group containing 1, 2 or 3 rings, two of which are in a “spiro” arrangement. Examples of suitable heterocyclyl groups include azetidine, tetrahydrofuranyl, tetrahydrothiophenyl, pyrrolidinyl, 2-oxopyrrolidinyl, pyrrolinyl, pyranyl, dioxolanyl, piperidinyl, 2-oxopiperidinyl, pyrazolinyl, imidazolinyl, thiazolinyl, dithiolyl, oxathiolyl, dioxanyl, dioxinyl, dioxazolyl, oxathiozolyl, oxazolonyl, piperazinyl, morpholino, thiomorpholinyl, 3-oxomorpholinyl, dithianyl, trithianyl and oxazinyl.

The term “heteroaryl” as used herein, represents a stable monocyclic, bicyclic or tricyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, isoindolyl, 1H,3H-1-oxoisoindolyl, benzotriazolyl, furanyl, thienyl, thiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinolinyl, thiazolyl, isothiazolyl, 1,2,3-triazolyl, 1,2,4-triazolyl, 1,2,4-oxadiazolyl, 1,2,4-thiadiazolyl, 1,3,5-triazinyl, 1,2,4-triazinyl, 1,2,4,5-tetrazinyl and tetrazolyl. Particular heteroaryl groups have 5- or 6-membered rings, such as pyrazolyl, furanyl, thienyl, oxazolyl, indolyl, isoindolyl, 1H,3H-1-oxoisoindolyl, isoxazolyl, imidazolyl, pyrazinyl, pyridazinyl, pyridinyl, pyrimidinyl, pyrrolyl, thiazolyl, isothiazolyl, 1,2,3-triazolyl, 1,2,4-triazolyl and 1,2,4-oxadiazolyl and 1,2,4-thiadiazolyl.

Alkyl, cycloalkyl, heterocyclyl, heteroaryl and aryl groups of the invention may be optionally substituted with 1 to 5 groups selected from OH, OC₁₋₆alkyl, Cl, Br, F, I, NH₂, NH(C₁₋₆alkyl), N(C₁₋₆alkyl)₂, SH, SC₁₋₆alkyl, CO₂H, CO₂C₁₋₆alkyl, CONH₂, CONH(C₁₋₆alkyl) or CON(C₁₋₆alkyl)₂.

As used herein, the term “divalent bridging group” refers to a radical that has a valence of two and is able to bind with two other groups. Examples of suitable divalent bridging groups include but are not limited to —(CH₂)ₜ— where t is an integer from 1 to 10, —O—, —S—, a divalent saturated or aromatic carbocyclic ring or a heterocyclic or heteroaromatic ring or a combination of such divalent and/or cyclic moieties. For example a saturated C₆ cyclic group would include —C₆H₁₀—, a C₆ aromatic group would include —C₆H₄—, a C₆ heterocyclic group would include

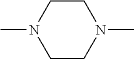

and a C₆ heteroaromatic would include

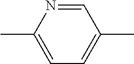

Other divalent bridging groups include alkylene groups (—CH₂—)₁ in which one or more carbon atoms have been replaced by NH, S, O,

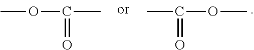

In a preferred embodiment the divalent bridging group is —(CH₂)ₜ— where t is an integer from 1 to 10, especially 1 to 6, more especially 3 to 6.

The compounds of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

The term “enclosed environment” refers to an indoor space bound by at least one wall, preferably four walls and a ceiling or roof. It does not include outdoor spaces but may include partially outdoor spaces such as covered balconies or covered decks bound by at least one wall and a ceiling or roof.

Uses of the Sanitizing Compositions of the Invention

The sanitizing compositions of the present invention have antimicrobial activity that is useful in applications sanitizing air in enclosed environments such as hospitals, nursing homes, schools, child care centres, gyms and sports clubs, shopping centres, homes, offices and other work places, and leisure centres such as cinemas, and other indoor areas where there is a risk of the spread of microbial infection.

Without wishing to be bound by theory, it is thought that the positive charges of the quaternary ammonium compounds attracts the negatively charged microbe particles in the air. When the microbes come in contact with the quaternary ammonium compounds their cell membranes are ruptured causing the microbes to die. The dead microbes are bonded to the quaternary ammonium compounds and are heavier, gently dropping out of the air and bonding to the surface they fall on. This prevents re-entry of the dead microbe into the air.

The sanitizing composition is effective against a wide range of pathogens which are either killed or inactivated following exposure of the composition. Pathogens which are killed or inactivated by the sanitizing compositions of the present invention include:

Gram Positive Bacteria

Bacillus sp. (vegetative cell), Corynebacterium diptheriae, Clostridium difficile, Enterococcus faecalis, Enterococcus hirae, Listeria monocytogenes, Micrococcus luteus, Micrococcus sp., Mycobacterium tuberculosis, Mycobacterium smegmatis, Propionibacterium acnes, Staphylococcus aureus, Staphylococcus epidermidis, Staphylococcus haemolyticus, Staphylococus hominis, Staphylococcus saprophyticus, Streptococcus faecalis, Streptococcus mutans, Streptococcus pneumonia and Streptococcus pyogenes.

Gram Negative Bacteria

Acinetobacter calcoaceticus, Aeromonas hydrophilia, Bacterioides fragilis, Citrobacter deversus, Citrobacter freundi, Enterobacter aerogenes, Enterobacter aglomerans, Enterobacter cloacae, Enterococcus, Escherichia coli, Escherichia coli O157:H7, Haemophilus influenza, Klebsiella oxytoca, Klebsiella pneumoniae, Klebsiella terriena, Legionella pneumophila, Morganella morganii, Proteus mirabilis, Proteus vulgaris, Pseudomonas aeruginosa, Pseudomonas fluorscens, Salmonella cholera suis, Salmonella typhi, Salmonella typhimurium, Salmonella enterica, Serratia liquifaciens, Serratia marcescens and Xanthomonas campestris.

Viruses

Adenovirus Type II & IV, Bovine Adenovirus Type I & IV, Murine Norovirus 1, Feline pneumonitis, Herpes Simplex Type I, Herpes Simplex Type II, HIV-1 (AIDS), Influenza A2 (Aichi), Influenza A2 (Asian), Influenza B, Influenza (H1N1), Mumps, Parinfluenza (Sendai), Rous Sarcoma, Reovirus Type I, Simian Virus 40, Vaccinia, MS2(bacteriophage) and PRD1 (bacteriophage).

Fungi, Algae, Mould, Yeast, Spores

Alterania alternata, Aphanizomenon sp., Aspergillus flavus, Aspergillus niger, Aspergillus sydowi, Aspergillus terreus, Aspergillus versicolor, Aspergillus vernicaria, Aureobasidium pullans, Candida albicans, Candida pseudotropocalis, Chaetomium globsum, Cladosporium cladosporioides, Chlorella vulgaris, Dreschslera australiensis, Epidermophyton sp., Epidermophyton floccosum, Gliomastix cerealis, Gloeophyllum trabeum, Microsporum sp., Microsporum audouinii, Monilia grisea, Oscillatoria, Penicillium chiysogenum, Pencillium commune, Penicillium funiculosum, Penicillium pinophilium, Penicillium variable, Phoma fimeti, Pithomyces chartarum, Poria placenta, Scenedesmus, Saccharonyces cerevisiae, Scolecobasidium humicola, Selenastrum sp., Trichoderma viride, Trichophyton interdigitale, Trichophyton maidson, Trichophyton mentogrophytes, Trichophyton rubrum and Trichophyton sp.

Protozoa Parasites

Cryptosporidium Parvum (oocysts).

In some embodiments, the composition is contained in a container that allows the composition to slowly evaporate. In other embodiments, the composition is contained in a container with a wick that allows the controlled dispersion of the composition into the air.

In some embodiments, the composition is dispersed from a container fitted into a dispersing device such as an atomizer, which may be wall mounted. In other embodiments, the composition is dispersed from a container fitted in an air purifying device that disperses the composition in an air flow or as a fog. Suitable air purifying devices are described in WO 2013/149285.

The invention will now be described with reference to the following Example which illustrates a preferred aspect of the invention. However, it is understood that the particularity of the following description of the invention is not to supersede the generality of the preceding description of the invention.

### EXAMPLES

### Example 1

### Air Sanitizing Compositions

A fragrant antimicrobial formulation was made with the following:

1. dipropylene glycol 58.70%

2. 1-Octadecanaminium-N,N-dimethyl-N-(3-trimethoxysilyl)propyl chloride 0.75%

3. polyaminopropylbiguanide 0.22%

4. benzalkonium chloride 0.15%

5. fragrance 40.0%

The ingredients were added in the order shown with constant mixing ensuring a homogeneous solution before addition of the next ingredient.

Fragrances used included one of citrus fragrance, aromatherapy fragrance, African Rain fragrance, and mango fragrance.

1. A sanitizing composition comprising:
i) an alkoxy silyl ammonium film-forming compound;
ii) a benzalkonium or benzethionium chloride,
iii) a polymeric biguanide, and
iv) a glycol carrier.

2. The sanitizing composition according to claim 1 wherein the alkylsilyl ammonium film-forming compound is selected from 3-trimethoxysilylpropyl-N,N-dimethyl-N-octadecyl ammonium chloride, 3-triethoxysilylpropyl-N,N-dimethyl-N-octadecyl ammonium chloride, 3-triethoxysilylpropyl-N,N-dimethyl-N-isodecyl ammonium chloride or 3-trimethoxysilylpropyl-N,N-dimethyl-N-isodecyl ammonium chloride.

3. The sanitizing composition according to claim 1 wherein the concentration of alkylsilyl ammonium film-forming compound is 0.1% to 1.5% w/w of the composition.

4. The sanitizing composition according to claim 1 wherein the benzalkonium or benzethonium chloride is present in an amount of 0.1% to 1.0% w/w of the composition.

5. The sanitizing composition according to claim 1 wherein the polymeric biguanide is a polyhexamethylene biguanide or polyaminopropylbiguanide.

6. The sanitizing composition according to claim 1 wherein the polymeric biguanide is present in an amount of 0.05% to 1.0% w/w of the composition.

7. The sanitizing composition according to claim 1 wherein the glycol is dipropylene glycol.

8. The sanitizing composition according to claim 1 wherein the glycol is present in the composition in the range of 40% to 99% w/w of the composition.

9. The sanitizing composition according to claim 1 further comprising a fragrance.

10. The sanitizing composition according to claim 9 wherein the fragrance is selected from essential oils, natural fragrances and synthetic fragrances and mixtures thereof.

11. The sanitizing composition according to claim 8 wherein the fragrance is present in an amount in the range of 20% to 60% w/w of the composition.

12. A method of sanitizing and/or deodorizing air in an enclosed environment comprising dispersing the composition according to claim 1 into the environment.

13. A method according to claim 12 wherein the composition is dispersed by evaporation.

14. A method according to claim 13 wherein the composition is dispersed from a device.

15. A method according to claim 14 wherein the device disperses the composition by atomisation, as a fog or in an airstream.

16. Use of the composition according to claim 1 as an air sanitizer and/or air deodorizer.
